(19) United States
(12) Patent Application Publication
(10) Pub. No.: US 2024/0285227 A1
(43) Pub. Date: Aug. 29, 2024

(54) SYSTEM, METHOD, AND DEVICES FOR DETERMINING STRUCTURES AND CHARACTERISTICS WITHIN A UTERUS

(71) Applicant: THE TRUSTEES OF COLUMBIA UNIVERSITY IN THE CITY OF NEW YORK, New York, NY (US)

(72) Inventors: CHRISTINE HENDON, Bronx, NY (US); HAIQIU YANG, New York, NY (US); ARIELLE JOASIL, North Chelmsford, MA (US); AIDAN THERIEN, Durham, NC (US)

(21) Appl. No.: 18/425,030

(22) Filed: Jan. 29, 2024

Related U.S. Application Data
(60) Provisional application No. 63/441,681, filed on Jan. 27, 2023.

(51) Int. Cl. A61B 5/00

(52) U.S. Cl. CPC A61B 5/4325; A61B 5/0075; A61B 5/0086

(57) ABSTRACT

Exemplary embodiments of the present disclosure are directed to systems, methods, and devices for determining at least one structure or at least one characteristic of a uterus. Exemplary embodiments of the present disclosure may include a probe, such as an endoscope, that includes a fiber bundle which includes a group of fibers which transmit one or more first electro-magnetic (EM) radiation to at least one portion of a uterus, and another group of fibers which receive one or more second EM radiations exiting the at least one portion which are based on the first EM radiations and a hyperspectral imaging camera obtaining at least one image data associated with the second EM radiations to determine the at least one of the structure or the at least one of the characteristic.

### CROSS REFERENCE TO RELATED APPLICATION(S)

This application relates to and claims the benefit of priority from U.S. Provisional Patent Application No. 63/441,681, filed on Jan. 27, 2023, the entire disclosure of which is incorporated herein by reference in its entirety.

### FIELD OF THE DISCLOSURE

The present disclosure relates generally to optical coherence tomography and near infrared spectroscopy probes for future real-time assessment of interventional and diagnostic procedures, and more particularly to systems, methods, and devices for determining structures and characteristics within a uterus using such technologies.

### BACKGROUND INFORMATION

Surgical methods for treatment of fibroids include myomectomy (removing the fibroid) or hysterectomy (removing the entire uterus organ). Currently, the indication of cancer only accounts for 10% of hysterectomies. Therefore, most women may only need conservative surgery, where lesions can be removed while preserving a woman's fertility. Women of color, historically, think that hysterectomy is the only option. As a result, they delay treatment resulting in worsening symptoms over time. In addition, this delay in treatment results in a more invasive intervention, which can include open surgery. Within the United States, further inequalities have been observed, where the rate of hysterectomies is much higher in the south versus the west coast or northeast and between African Americans and Caucasians. These trends are not unique to the United States. Technology development, access, and innovative technologies are all needed to level the playing field, to ensure that uterine (and fertility) preserving technologies are accessible to all that qualify.

Thus, it may be beneficial to provide exemplary optical endoscopes to enable future diagnosis of uterine pathologies at earlier stages, which can overcome at least some of the deficiencies described herein above.

### SUMMARY OF EXEMPLARY EMBODIMENTS

The following is intended to be a brief summary of the exemplary embodiments of the present disclosure, and is not intended to limit the scope of the exemplary embodiments.

According to certain exemplary embodiments of the present disclosure, exemplary systems, methods, and devices can be provided which can include probes for determining structures and characteristics within a uterus. These probes may be endoscopes or any other type of probe. They may include a fiber bundle and a hyperspectral imaging camera.

In some exemplary embodiments of the present disclosure, exemplary systems, methods, and devices can be provided which can include, e.g., probes that can be configured to not contact a surface of the uterus that is being analyzed. According to further embodiments of the present disclosure, the probes can be configured to contact a uterine surface. Further, the exemplary probes can rely on and/or utilize a spectral contrast to identify structures and characteristics of a uterus. In certain embodiments, the fiber bundle of the probes can be configured to emit near infrared wavelengths. Additionally, for example, the fiber bundle can emit both near infrared wavelengths and visible wavelengths. and/or the fiber bundle can include 1000 fibers or more.

In some still further exemplary embodiments of the present disclosure, exemplary methods for determining structures and characteristics in a uterus can be provided, whereas it is possible to illuminate a uterine surface with a fiber bundle in a probe and capturing, e.g., by a hyperspectral imaging camera in the probe, image data of the illuminated uterine surface,

These and other objects, features and advantages of the exemplary embodiments of the present disclosure will become apparent upon reading the following detailed description of the exemplary embodiments of the present disclosure, when taken in conjunction with the accompanying claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an exemplary illustration of a contact Near Infrared (NIR) spectroscopy system according to an exemplary embodiment of the present disclosure;

FIG. 2 is an exemplary illustration of a non-contact probe system for surgical guidance, biopsy guidance, and/or surveillance according to an exemplary embodiment of the present disclosure;

FIG. 3 is a set of diagrams providing a flow diagram of an exemplary method for assessing the tissue architecture from OCT en face, projection and/or hyperspectral endoscopic images according to an exemplary embodiment of the present disclosure;

FIG. 4 is an exemplary flow diagram of a method providing a linear tissue classification model according to an exemplary embodiment of the present disclosure;

FIG. 5A is an exemplary graph of a spectral shape of the relative reflectance of spectral composition of uterine pathologies collected from a contact based near infrared spectroscopy probe according to an exemplary embodiment of the present disclosure;

FIG. 5B is an exemplary graph of the spectral shape of a derivative of the relative reflectance of spectral composition of uterine pathologies collected from a contact based near infrared spectroscopy probe according to an exemplary embodiment of the present disclosure;

FIGS. 5C-5E are exemplary graphs illustrating optical indices with significant difference between areas of non-fibroid and fibroids according to an exemplary embodiment of the present disclosure;

FIG. 5F is a set of exemplary images of example maps of uterine tissue with non-fibroid areas according to an exemplary embodiment of the present disclosure;

FIG. 5G is a set of exemplary images of exemplary maps of the uterine tissue with a fibroid area according to an exemplary embodiment of the present disclosure;

FIG. 6 is a set of exemplary graphs illustrating a spectral analysis according to an exemplary embodiment of the present disclosure;

FIG. 7A is an exemplary hyperspectral image of a uterine specimen according to an exemplary embodiment of the present disclosure;

FIG. 7B is an exemplary graph of visible and near infrared (NIR) spectra of a fibroid and an adjacent normal according to an exemplary embodiment of the present disclosure;

FIG. 8A is an exemplary endoscopic image of the uterine specimen using a fiber bundle according to an exemplary embodiment of the present disclosure;

FIG. 8B is an exemplary graph of the visible and near infrared spectra of the fibroid and the adjacent normal according to another exemplary embodiment of the present disclosure;

FIG. 9 is a set of exemplary images of human uterine specimens obtained with a hyperspectral imaging camera according to an exemplary embodiment of the present disclosure.

FIG. 10 is a series of exemplary OCT images of tumors from 4 samples according to an exemplary embodiment of the present disclosure;

FIG. 11A is an exemplary projection OCT image according to an exemplary embodiment of the present disclosure;

FIG. 11B is an exemplary histology image corresponding to the OCT image of FIG. 11A according to an exemplary embodiment of the present disclosure;

FIG. 11C is an exemplary graph showing that the concentration parameter b obtained from fiber orientation analysis can distinguish a tumor from normal uterine specimens, according to an exemplary embodiment of the present disclosure; and

FIG. 12 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The following description of exemplary embodiments provides non-limiting representative examples referencing numerals to particularly describe features and teachings of different exemplary aspects and exemplary embodiments of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of embodiments, would be understood to be consistent with an application of the exemplary embodiments of the present disclosure.

### Exemplary Optical Coherence Tomography (OCT) Imaging Facilitates Real-Time Pathology Assessment

OCT facilitates subsurface imaging of depths of, e.g., 1-2 mm in tissue with high spatial resolution in three dimensions and high sensitivity in vivo. Fiber-based OCT systems can be incorporated into catheters to image internal organs. These features have made OCT a powerful tool for medical imaging and has revolutionized fields such as ophthalmology^{1,2}, cardiology³⁻⁵ and dermatology⁶.

The pathophysiology of fibroids highlights areas where OCT imaging can detect changes in tissue architecture and optical properties. The endometrium, the innermost layer of the uterus, is composed of both glandular epithelium and stromal cells⁷. When cancer originates from glandular epithelial cells, the glands begin to crowd and form “back-to-back”, or cystic patterns⁸. The ratio of glands to stroma increases as the cancer grows⁸. Mesenchymal tumors originate from smooth muscle cells in the myometrium⁹, which provide structure and function to the uterus. Leiomyosarcomas exhibit less collagen than leiomyomas, due to the invasive growth of the cancer cells. Microscopically, the cancer cells are pleomorphic with large nuclei and several mitoses^{10,11}, often accompanied by necrosis and hemorrhages¹¹. Macroscopically, leiomyosarcomas are also softer and homogeneous in appearance¹⁰⁻¹³. To support the growth of tumors, increased blood supply is necessary. Blood vessels may appear differently in diseased tissue when compared to normal tissue¹⁴. The amount, pattern, and thickness of blood vessels can help determine the disease that is present¹⁵⁻¹⁸. All of these features can result in changes in optical imaging and spectral contrast. Quantitative measurements of optical uterine properties can provide a foundation for disease detection and therapy design. To date, there is limited to no knowledge of uterine optical properties.

### DISCUSSION

Optical imaging is widely used in gynecologic surgery, including laparoscopy and endoscopy. Comprehensive measurement and characterization of optical properties of the uterus, according to exemplary embodiments of the present disclosure, can assist in providing technologies to (1) characterize how optical properties change with age, (2) identify smaller fibroids not visualized on MRI and ultrasound and (3) aid in differentiating fibroids from leiomyosarcomas.

### Exemplary Optical Imaging of Uterine Fibroids, Cancer, and Tissue Architecture

Within the uterus, remodeling due to disease causes changes in the composition of fibrosis, scar, collagen, and smooth muscle. Changes in tissue architecture can be visualized as changes in collagen and muscle fiber orientation and disarray¹⁹. Systems, methods, devices and computer-accessible medium according to exemplary embodiments of the present disclosure can be used to observe similar changes to uterine tissue collected from, e.g., 12 patients with a range of pathology. Increased density of collagen can result in an increased intensity within optical coherence tomography images according to exemplary embodiments of the present disclosure, and visualization of the collagen fiber bundles in two and three dimensions. Using such systems, methods, devices and computer-accessible medium according to exemplary embodiments of the present disclosure, regular fiber organization can be observed and quantified within areas of normal tissue, which may no longer be apparent for regions of tumors (cancerous, fibroids, and other benign tumors). Preliminary imaging of seedling fibroid tumors can be characterized by bright areas within OCT volumes, surrounded by circumferential fibers. Fiber orientation analysis, according to exemplary embodiments of the present disclosure, can show that the gradients within the normal samples are less concentrated than the tumor samples (tumor: 0.2206±0.1733 rad, normal: 0.1506±0.1464 rad), highlighting the remodeling of the collagen fiber architecture due to the presence of tumors.

In addition, systems, methods, devices and computer-accessible medium according to exemplary embodiments of the present disclosure can indicate statistical differences (p<0.05) between texture and statistical features (variance and homogeneity) extracted from standard deviation projections of tumor and normal samples. Collecting data from patients with diverse backgrounds (i.e., birth control status, age, parity) that impact tissue architecture can ensure that the initial changes in tissue scattering (intensity) and fiber organization are key in distinguishing tumor and normal samples. systems, methods, devices and computer-accessible medium according to exemplary embodiments of the present disclosure can also utilize exemplary procedures that identify optical signatures and developing machine learning and deep learning classification procedures classifying remodeling and disease within optical coherence tomography images for fibrosis^{5,20}, adipose tissues²¹, breast cancer²²⁻²⁵.

### Exemplary Image Analysis: Tissue Architecture Characterization

Systems, methods, devices and computer-accessible medium according to exemplary embodiments of the present disclosure can utilize exemplary computational procedures for examining collagen architecture in OCT images of cardiac, cervical, and uterine tissue^{18,124}. According to various exemplary embodiments of the present disclosure, the images can be passed through a preprocessing regime comprising a wedge filter, median filter, denoising, and homomorphic filtering followed by intensity mapping.

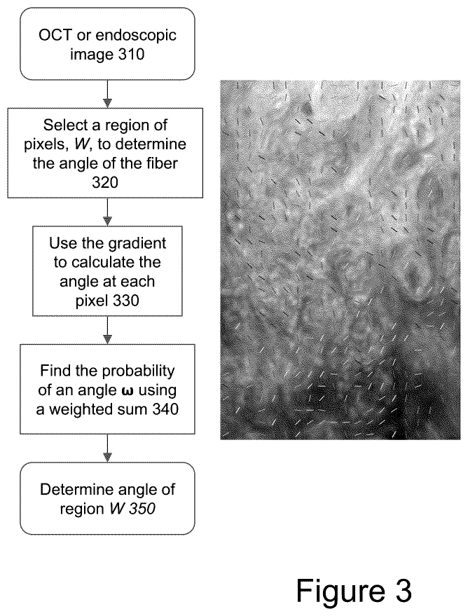

FIG. 3 shows a flow diagram of an exemplary method according to an exemplary embodiment of the present disclosure which can be used to provide and/or obtain fiber orientation architecture measurements and associated image utilized and/or generated thereby according to exemplary embodiments in the B-scan and enface direction after pre-processing, which can enhance the visualization of collagen fiber bundles. For example, in step 310 of FIG. 3, OCT or endoscopic image is obtained. In step 320, the received image divided 320 smaller sub regions W, and a region of pixels W is selected to determine the angle of orientation of the fiber. In step 330, the gradient are calculated and angles are measured from the images in the enface direction after pre-processing. In step 340, the probability of an angle ω is determined using a weighted sum. Further, in step 350, angle of region W is determined and/or probability of an angle is determined using a weighted sum. This can be done for OCT en face, projection and/or hyperspectral endoscopic images.

According to another exemplary embodiments of the present disclosure, a sobel filter can be used to identify the gradient direction of the collagen fibers within the enface (ϕ) and B-scan (θ) directions to describe a three-dimensional orientation. To quantify how aligned the fibers are, the probability distribution P(x) is calculated within a subregion of the image.

$$P ( x ) = \frac{{e}^{b c o {s ( x - θ )}^{■}}}{2 π {{I}_{0} ( b )}_{{■}_{■}}} ,$$

where b is the concentration parameter, θ is the fiber bundle direction, and I₀(b) is the modified Bessel function of the first kind at order 0¹²⁵. The dispersion is inversely proportional to the concentration parameter. According to exemplary embodiments of the present disclosure, aligned fibers have a low dispersion and high concentration parameter. Fibers with disarray have a high dispersion and low concentration parameter. In addition, the patterns, shapes, and number of blood vessels and glands within OCT B-scans and en face slices can be quantified. The Hough transform can be used with the systems, methods, devices and computer-accessible medium according to exemplary embodiments of the present disclosure to locate circles and quantify the number and size of glands and blood vessels present.

Data according to exemplary embodiments shows that the mean±SD of concentration, b, is 0.22±0.17 rad for fibrous tissues and 0.15±0.15 rad for normal tissues.

### Exemplary Tissue Classification: Random Forest and Logistic Regression

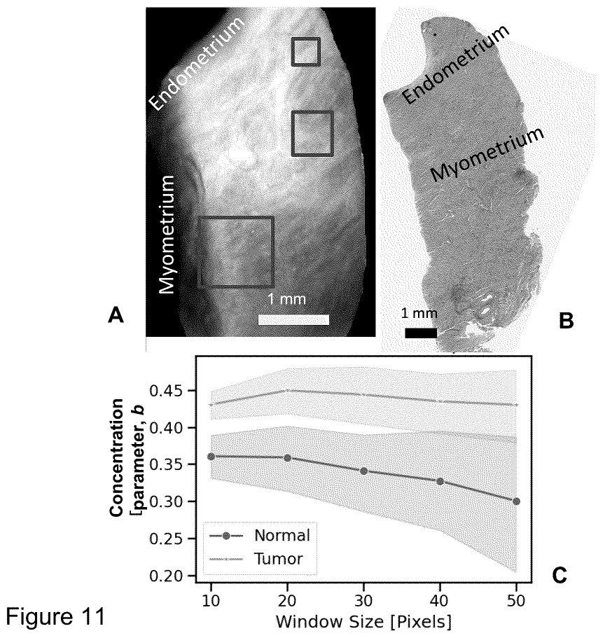

Exemplary tissue classification models according to exemplary embodiments can be developed using optical characterization. Previous features extracted from heart and breast cancer optical imaging datasets can be first assessed for texture, fiber orientation and dispersion, and attenuation coefficient. As shown in FIG. 11, exemplary rectangular patches can be extracted, according to exemplary embodiments, and texture and statistical features (such as, e.g., kurtosis, skewness, entropy, correlation, mean, max, variance, energy, spectral shape, theta, phi, concentration parameter, number of circles, size of circles, attenuation, backscattering, absorption, penetration depth, etc.) can be calculated. These features can act as inputs in exemplary embodiments for linear and ensemble classification models such as logistic regression and random forest, respectively.

### Exemplary Tissue Classification: Weakly Supervised Learning for Detection of Fibroids

A superior performance of the exemplary systems, methods and computer-accessible medium providing deep learning for image classification can be tied to the availability of large-scale datasets, which requires tremendous effort in the annotation process. To lessen the need for pixel-wise labels, systems, methods and computer-accessible medium according to exemplary embodiments of the present disclosure can provide a deep-learning framework for fibroid segmentation via weakly supervised techniques. This framework has been previously evaluated on a human cardiac OCT dataset with comparable results with fully supervised deep learning models¹²⁶. Such exemplary systems, methods and computer-accessible medium can provide pixel-wise fibroid segmentation through the use of only image-level annotations. Further, systems, methods and computer-accessible medium according to the exemplary embodiments of the present disclosure can combine class active mapping (CAM) with super-pixel segmentation to effectively address the tissue segmentation challenges caused by irregular shapes and blurry boundaries in the OCT images. The systems, methods and computer-accessible medium according to the exemplary embodiments can include, e.g., separate modules, such as, e.g., a pseudo label generation and a segmentation network training. In the pseudo label generation module, pixel-wise pseudo annotations can be generated by the integration of CAM and super-pixel methods.

### Further Discussion

Standard optical imaging within gynecology is based on white light or fluorescence endoscopy and laparoscopy. The tissue architecture and composition (smooth muscle, collagen, and adipose) changes in the presence of fibroids and cancer. Thus, spectral contrast, according to exemplary embodiments, can be a powerful addition to current endoscopes for gynecology.

### Exemplary Data

The effectiveness of multi-spectral catheter have been demonstrated^{21,26-30} and endoscopic imaging³¹ in resolving substrates of high interest within the heart, for cardiac radiofrequency ablation therapy. Systems, methods and computer-accessible medium according to exemplary embodiments of the present disclosure can rely on and/or utilize various near infrared spectroscopy (NIRS) systems.

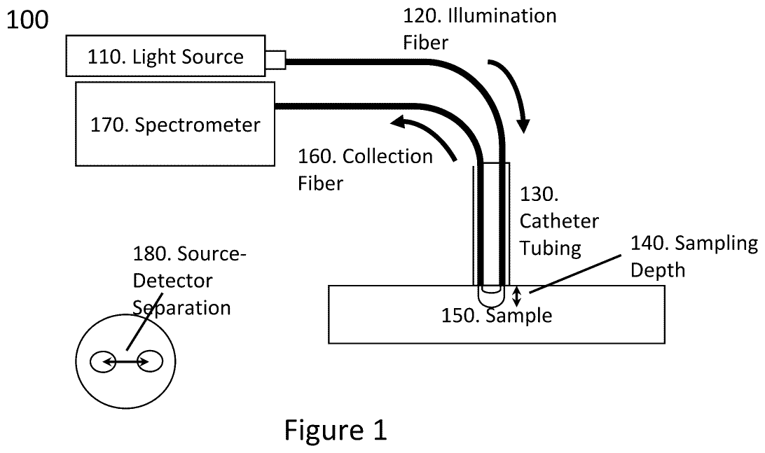

FIG. 1 illustrates an exemplary near infrared spectroscopy system 100 that may be used for tissue characterization according to the exemplary embodiment of the present disclosure. The exemplary system 10 can include a light source 110. The light from the light source 110 can be delivered to sample tissue 150, through an illumination fiber 120 where the illumination fiber may run through catheter tubing 130. The reflected signal can be obtained from a particular sampling depth and volume 140, determined by separation distance 180, between the illumination fiber 120, and the collection fiber 160, where the collection fiber 160 serves as an input to spectrometer 170. Diffuse light can be collected from a separate multimode fiber, where the distance between illumination and collection fibers can be optimized to sample depths 5-7 mm.

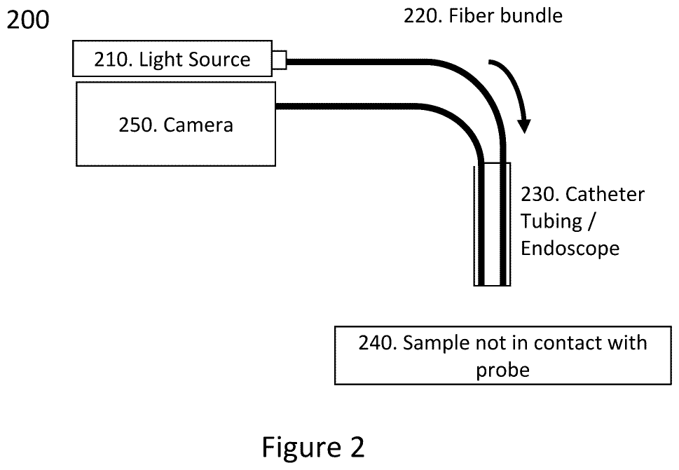

FIG. 2 illustrates an exemplary non-contact probe system 200 for surgical guidance, biopsy guidance or surveillance. The light source 210 can be a broadband light source, LEDs, lasers, etc. The light source 210 can be coupled into a fiber bundle 220 and/or endoscope 230 to allow for large field of view imaging, with or without contact to the tissue 240. Within the accessory port of the endoscope 230, a single mode fiber can be used for OCT imaging. A tracking sensor for 3D positioning can enable stitching of mosaic OCT and camera images. The camera 250 can be a near infrared camera, visible RGB camera, and/or hyperspectral imaging camera. The system can work with our without polarizers placed at the distal tip of the probe and before the camera.

An exemplary endoscopic multi-spectral imaging system according to one exemplary embodiment of the present disclosure can include, e.g., an endoscope, a microcontroller, a camera, and requisite collimating, focusing, and filtering optics. LED light (LED sources: 940 nm, 810 nm, 625 nm, 530 nm, and 450 nm) can be coupled into the endoscope lighting port using a custom lens and dichroic filter assembly. The endoscope can be flexible and, e.g., 0.75 mm in diameter, has a viewing angle of, e.g., 70°, and can contain, e.g., 10,000 fibers. The viewing port of the endoscope can be connected to the CMOS camera (Hamamatsu Flash 4.0LT, Hamamatsu City, Japan). A second contact based NIRS can collect diffusely scattered light and is recorded onto a spectrometer. The source fiber can be connected to a broadband light source (HL-2000HP, Ocean Optics Inc, Dunedin, FL) and can illuminate light onto the tissue surface. The detection fiber can collect diffusely backscattered light, which can then be analyzed by the spectrometer (e.g., 600-1000 nm) (e.g., C9405CB, Hamamatsu, Bridgewater, NJ). According to exemplary embodiments of the present disclosure, exemplary results of spectral shape features between normal and uterine tumors show statistical differences (FIG. 11C, n=23 samples).

FIG. 11 provides representative examples of optical image features. Images used can be images from the hyperspectral imaging camera, endoscope or OCT. These include images generated from multiple frames, en face images, of spectral challenges. Features can be extracted from the whole image or from small regions (patches/windows) to enabled a spatial analysis of features. Features can include optical property based features (fitting attenuation, absorption, and scattering coefficients), optical indices, texture features and statistical moments. In addition, fiber orientation to assess tissue architecture can be carried out. FIG. 11A shows an example of a projection OCT image, FIG. 11B is the corresponding exemplary histology image. Exemplary FIG. 11C shows that the concentration parameter, b, obtained from fiber orientation analysis can distinguish tumor from normal uterine specimens. These features can then be used to classify tumors and normal classes using a CNN. Different inputs may include B-scans; patches; multi-channel projections; multi-channel projections, hyperspectral imaging channels, and spectral dependent maps.

### Exemplary Near Infrared Spectroscopy (NIRS) System

Systems, methods and computer-accessible medium according to exemplary embodiments of the present disclosure can provide various types of NIRS probes, modeled after advancements in providing various catheter probes for the heart. This can include, e.g., contact and non-contact-based probes. The exemplary design parameters of contact probes for exemplary embodiments can include the number of fibers, fiber core diameter, numerical aperture, distance between fibers, and spectral range of the illumination source. Exemplary embodiments of the present disclosure can conduct Monte Carlo simulations to evaluate the sampling volume for various optical configurations. exemplary embodiments can prototype catheters with multiple source detection separations (0.5 mm-4 mm). With the increasing separation of the fibers, the light is able to probe deeper into the tissue. The exemplary contact probe according to an exemplary embodiment of the present disclosure is shown in FIG. 1, and described herein above. It is noted that such probe is not limited to the probe shown in FIG. 1, and other probes (and components thereof) can be implemented in accordance with the exemplary embodiments of the present disclosure.

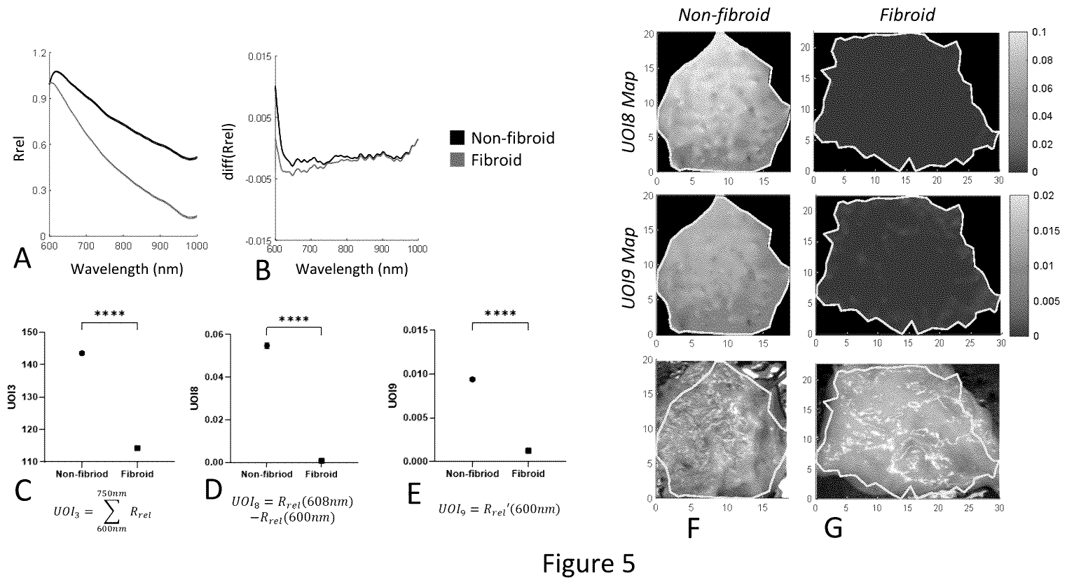

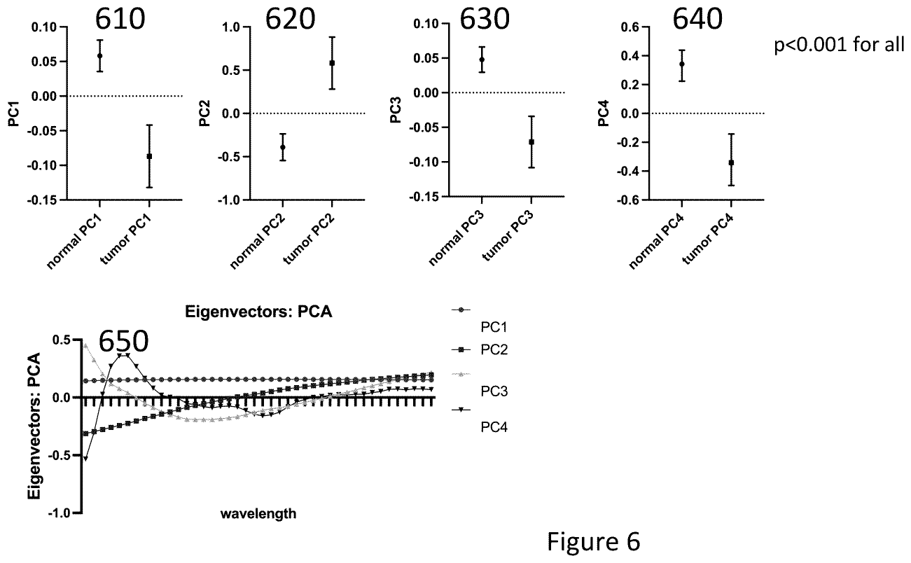

FIG. 5 provides examples of contact based near infrared spectroscopy probe for assessing the spectral composition of uterine pathologies. There can be a difference in spectral shape of the relative reflectance (FIG. 5A) and derivative of the relative reflectance spectra (FIG. 5B). Spectral shape can be quantified with optical indices, optical property extraction (absorption coefficient and scattering coefficient) and chromophore composition. Examples of optical indices are shown in FIGS. 5C-5E, with significant difference between areas of non-fibroid and fibroids. As the contact probe is translated across the surface of the tissue, with tracking of the 3D position of the probe, maps can be generated of features, including optical indices, classes, optical properties. Example maps of uterine tissue with non-fibroid areas (FIG. 5F) and an area with a fibroid (FIG. 5G).

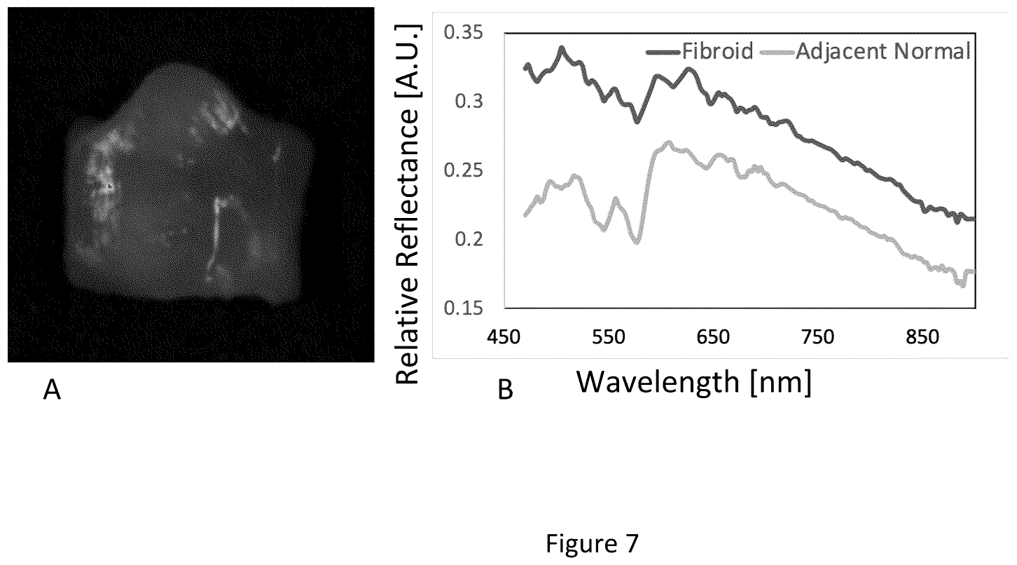

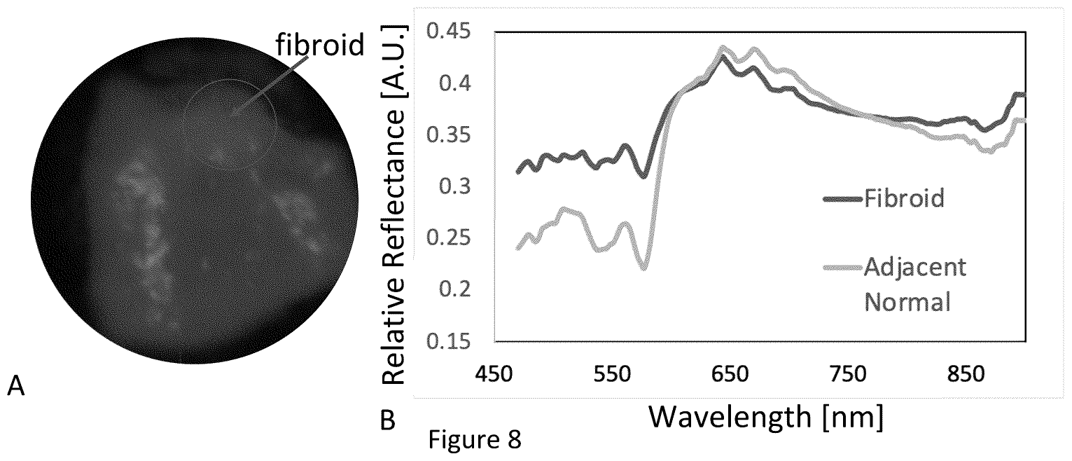

The non-contact NIR spectral-endoscope, according to exemplary embodiments of the present disclosure, can be provided which can include a fiber bundle with a minimum of 10,000 fibers. Exemplary systems can incorporate the same or similar broadband source used within the contact NIRS system and a hyperspectral imaging camera. In exemplary embodiments, the backscattered light can be collected by a hyperspectral imaging camera. In certain exemplary embodiments of the present disclosure, cross polarizers can be placed in front of the camera and the sources to minimize back reflectance. The exemplary non-contact probe according to an exemplary embodiment of the present disclosure is shown in FIG. 2, and described herein above. It is noted that such probe is not limited to the probe shown in FIG. 2, and other probes (and components thereof) can be implemented in accordance with the exemplary embodiments of the present disclosure. FIG. 7A shows an exemplary hyperspectral image of a uterine specimen according to exemplary embodiments of the present disclosure. Further, FIG. 8A shows a representative exemplary endoscopic image using a fiber bundle of a uterine specimen. Endoscopic images recorded with a hyperspectral imaging camera can produce a white light image, typically observed with surgery in addition to spectral information for each pixel. FIGS. 7B and 8B are representative examples of visible and near infrared spectra of a fibroid and adjacent normal.

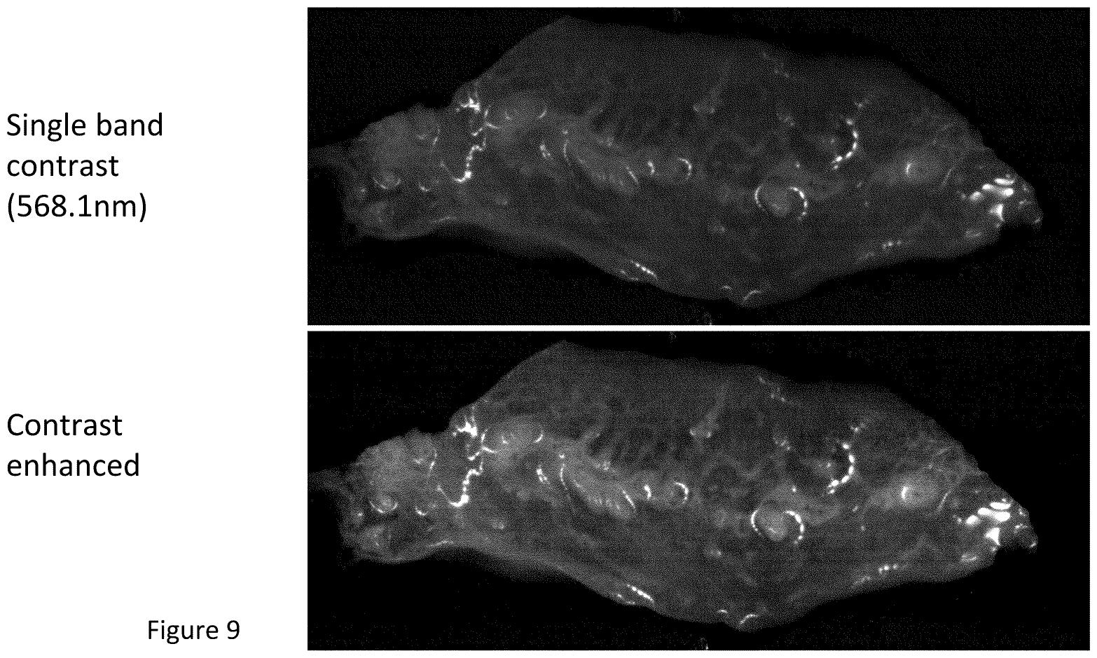

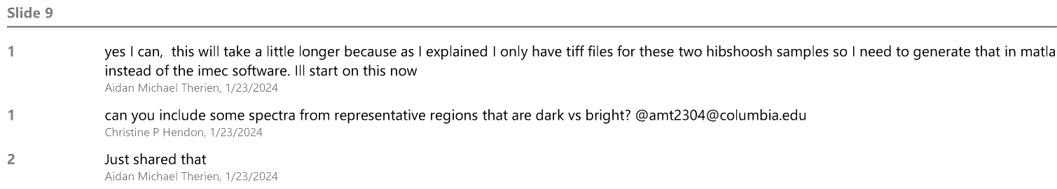

FIG. 9 shows a set of exemplary images of human uterine specimens obtained with a hyperspectral imaging camera, according to an exemplary embodiment of the present disclosure. For example, one image in FIG. 9 illustrates a single band contrast. Another image of FIG. 9 is shown with a contrast enhanced. Within such exemplary image, collagen fiber architecture is highlighted.

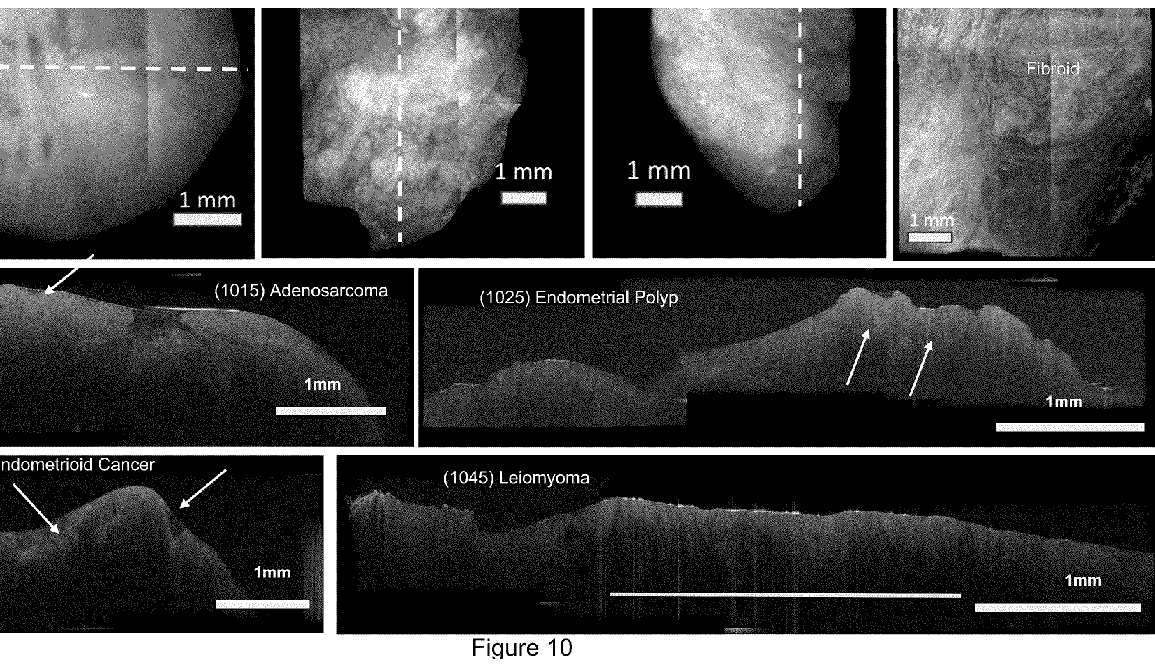

FIG. 10 provides exemplary OCT images of tumors from 4 samples according to exemplary embodiments of the present disclosure. (1) Standard deviation projection of the samples along with (2) B-scans that showcase features of the tumor. (a) (1010) adenosarcoma with sarcomatous overgrowth (malignant), (1020) endometrial polyp (benign), (1030) endometrioid (malignant), (1040) leiomyoma (benign). (1015) Arrows pointing to regions of low reflectivity are glands. (1025, 1035) Arrows are collagen fiber bundles. (1045) Transition from myometrium to aligned collagen fiber bundles surrounding the disorganized contents of the fibroid are highlighted by the yellow line.

### Exemplary Imaging Protocol and Processing

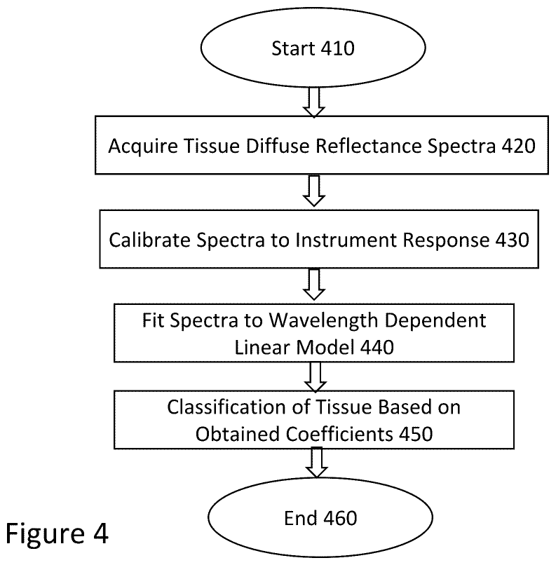

Comprehensive imaging can be acquired using the exemplary method/procedure according to exemplary embodiments of the present disclosure, by obtaining spectra at multiple points (e.g., contact probe) or multiple images (e.g., endoscope). According to exemplary embodiments, NIRS measurements can be calibrated and converted into relative reflectance spectrums (Rrel) by using a phantom measurements of known optical properties, then normalized by the reflectance at a single wavelength, such as 600 nm. Exemplary embodiments can derive optical indices to highlight the spectral shape differences between fibroid and non-fibroid areas. In addition, texture and fiber architecture image features (such as, e.g., kurtosis, skewness, entropy, correlation, mean, max, variance, energy, spectral shape, theta, phi, concentration parameter, number of circles, size of circles, attenuation, backscattering, absorption, penetration depth, etc.) can be extracted from endoscopic images. An exemplary method/procedure according to the exemplary embodiments of the present disclosure to perform such calibration is shown in FIG. 4. For example, as shown in FIG. 4, in step 410, the calibration process may begin. In step 420, tissue diffuse reflectance spectra may be acquired. In step 430, a spectra to instrument response can be calibrated. In step 440, the spectra may be fit to a wavelength dependent linear model. In step 450, tissue based on obtained coefficients may be classified. In step 460, the process may end.

### Exemplary Image Classification

Convolutional neural networks (CNNs) have achieved superior performance in various machine learning fields^{32,33}. However, CNNs cannot be easily implemented on graph-structured data with non-Euclidean structured inputs. To overcome this challenge, systems, methods and computer-accessible medium according to exemplary embodiments of the present disclosure can provide and/or include a graph convolutional network (GCN) model to identify the fibroid using the NIRS contact probe. The inputs to the GCN model can include the feature vectors for the sampled points and an adjacency matrix that defines the points' spatial connections. The adjacency matrix can be calculated based on the Euclidean distances between different points. In exemplary embodiments, the feature vector can be fed into a GCN composed of 3 hidden layers with 32, 16, and 8 channels, respectively. The final layer, according to exemplary embodiments of the present disclosure, can be a single node with a sigmoid activation function whose output is the probability of the input point lying on a fibroid. Endoscopic images obtained and/or generated exemplary embodiments can utilize random forest, where the features vector includes spectral shape and image features (such as, e.g., kurtosis, skewness, entropy, correlation, mean, max, variance, energy, spectral shape, theta, phi, concentration parameter, number of circles, size of circles, attenuation, backscattering, absorption, penetration depth, etc.)

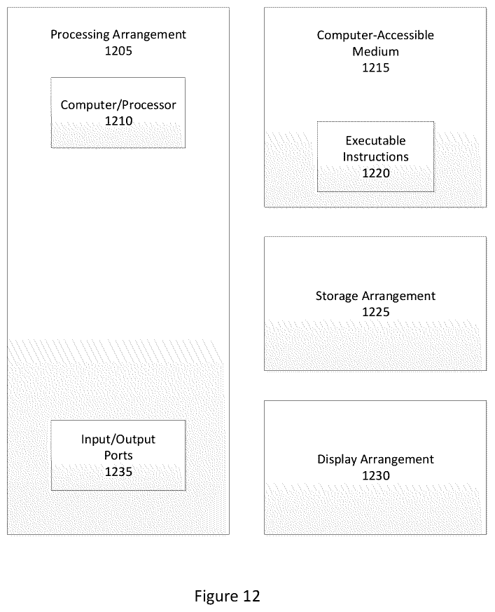

FIG. 12 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1205. Such processing/computing arrangement 1205 can be, for example entirely or a part of, or include, but not limited to, a computer/processor 1210 that can include, for example one or more microprocessors, and use instructions stored on a computer-accessible medium (e.g., RAM, ROM, hard drive, or other storage device).

As shown in FIG. 12, for example a computer-accessible medium 1215 (e.g., as described herein above, a storage device such as a hard disk, floppy disk, memory stick, CD-ROM, RAM, ROM, etc., or a collection thereof) can be provided (e.g., in communication with the processing arrangement 1205). The computer-accessible medium 1215 can contain executable instructions 1220 thereon. In addition or alternatively, a storage arrangement 1225 can be provided separately from the computer-accessible medium 1215, which can provide the instructions to the processing arrangement 1205 so as to configure the processing arrangement to execute certain exemplary procedures, processes, and methods, as described herein above, for example.

Further, the exemplary processing arrangement 1205 can be provided with or include an input/output ports 1235, which can include, for example a wired network, a wireless network, the internet, an intranet, a data collection probe, a sensor, etc. As shown in FIG. 12, the exemplary processing arrangement 1205 can be in communication with an exemplary display arrangement 1230, which, according to certain exemplary embodiments of the present disclosure, can be a touch-screen configured for inputting information to the processing arrangement in addition to outputting information from the processing arrangement, for example. Further, the exemplary display arrangement 1230 and/or a storage arrangement 1225 can be used to display and/or store data in a user-accessible format and/or user-readable format.

According to exemplary embodiments of the present disclosure, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended paragraphs. Although specific terms are employed herein, they are used in a generic and descriptive sense only and not for purposes of limitation.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced and identified herein are incorporated herein by reference in their entireties.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

This written description uses examples to disclose certain implementations of the disclosed technology, including the best mode, and also to enable any person skilled in the art to practice certain implementations of the disclosed technology, including making and using any devices or systems and performing any incorporated methods.

### EXEMPLARY REFERENCES

1. Fujimoto J G, Pitris C, Boppart S A, Brezinski M E. Optical coherence tomography: an emerging technology for biomedical imaging and optical biopsy. Neoplasia. 2000 January-April; 2(1-2):9-25. PMCID: PMC1531864
2. Hee M R, Puliafito C A, Wong C, Duker J S, Reichel E, Rutledge B, Schuman J S, Swanson E A, Fujimoto J G. Quantitative assessment of macular edema with optical coherence tomography. Arch Ophthalmol. 1995 August; 113(8):1019-1029. PMID: 7639652
3. Aguirre A D, Arbab-Zadeh A, Soeda T, Fuster V, Jang I K. Optical Coherence Tomography of Plaque Vulnerability and Rupture: JACC Focus Seminar Part 1/3. J Am Coll Cardiol. 2021 Sep. 21; 78(12):1257-1265. PMCID: PMC9851427
4. Terashima M, Kaneda H, Suzuki T. The role of optical coherence tomography in coronary intervention. Korean J Intern Med. 2012 March; 27(1):1-12. PMCID: PMC3295975
5. Gan Y, Tsay D, Amir S B, Marboe C C, Hendon C P. Automated classification of optical coherence tomography images of human atrial tissue. J Biomed Opt. 2016 October; 21(10):101407. PMCID: PMC5995000
6. Steiner R, Kunzi-Rapp K, Scharffetter-Kochanek K. Optical Coherence Tomography: Clinical Applications in Dermatology. Med Laser Appl. 2003 Jan. 1; 18(3):249-259.
7. Masuda A, Katoh N, Nakabayashi K, Kato K, Sonoda K, Kitade M, Takeda S, Hata K, Tomikawa J. An improved method for isolation of epithelial and stromal cells from the human endometrium. J Reprod Dev. 2016 Apr. 22; 62(2):213-218. PMCID: PMC4848580
8. Kim J J, Kurita T, Bulun S E. Progesterone action in endometrial cancer, endometriosis, uterine fibroids, and breast cancer. Endocr Rev. 2013 February; 34(1):130-162. PMCID: PMC3565104
9. Santos P, Cunha T M. Uterine sarcomas: clinical presentation and MRI features. Diagn Interv Radiol. 2015 January-February; 21(1):4-9. PMCID: PMC4463355
10. Brown L. Pathology of uterine malignancies. Clin Oncol. 2008 August; 20(6):433-447. PMID: 18499412
11. Prayson R A, Hart W R. Pathologic considerations of uterine smooth muscle tumors. Obstet Gynecol Clin North Am. 1995 December; 22(4):637-657. PMID: 8786875
12. Robboy S J, Bentley R C, Butnor K, Anderson M C. Pathology and Pathophysiology of Uterine Smooth-Muscle Tumors. Environ Health Perspect. [National Institute of Environmental Health Sciences, Brogan & Partners]; 2000; 108:779-784.
13. Artioli G, Wabersich J, Ludwig K, Gardiman M P, Borgato L, Garbin F. Rare uterine cancer: carcinosarcomas. Review from histology to treatment. Crit Rev Oncol Hematol. 2015 April; 94(1):98-104. PMID: 25468677
14. Nagy J A, Chang S H, Dvorak A M, Dvorak H F. Why are tumour blood vessels abnormal and why is it important to know? Br J Cancer. 2009 Mar. 24; 100(6):865-869. PMCID: PMC2661770
15. Nucci M R. Practical issues related to uterine pathology: endometrial stromal tumors. Mod Pathol. 2016 January; 29 Suppl 1:592-103. PMID: 26715176
16. Oliva E, Young R H, Clement P B, Bhan A K, Scully R E. Cellular benign mesenchymal tumors of the uterus. A comparative morphologic and immunohistochemical analysis of 33 highly cellular leiomyomas and six endometrial stromal nodules, two frequently confused tumors. Am J Surg Pathol. 1995 July; 19(7):757-768. PMID: 7793473
17. Oliva E. Cellular mesenchymal tumors of the uterus: a review emphasizing recent observations. Int J Gynecol Pathol. 2014 July; 33(4):374-384. PMID: 24901397
18. Tahlan A, Nanda A, Mohan H. Uterine adenomyoma: a clinicopathologic review of 26 cases and a review of the literature. Int J Gynecol Pathol. 2006 October; 25(4):361-365. PMID: 16990713
19. McLean J P, Fang S, Gallos G, Myers K M, Hendon C P. Three-dimensional collagen fiber mapping and tractography of human uterine tissue using OCT. Biomed Opt Express. 2020 Oct. 1; 11(10):5518-5541. PMCID: PMC7587264
20. Yao X, Gan Y, Ling Y, Marboe C C, Hendon C P. Multicontrast endomyocardial imaging by single-channel high-resolution cross-polarization optical coherence tomography. J Biophotonics. 2018 April; 11(4):e201700204. PMCID: PMC6186148
21. Singh-Moon R P, Park S Y, Song Cho D M, Vaidya A, Marboe C C, Wan E Y, Hendon C P. Feasibility of near-infrared spectroscopy as a tool for anatomical mapping of the human epicardium. Biomed Opt Express. 2020 Aug. 1; 11(8):4099-4109. PMCID: PMC7449747
22. Yu Gan, Xinwen Yao, Chang E, Bin Amir S, Hibshoosh H, Feldman S, Hendon C P. Comparative study of texture features in OCT images at different scales for human breast tissue classification. Conf Proc IEEE Eng Med Biol Soc. 2016 August; 2016:3926-3929. PMCID: PMC6180913
23. Mojahed D, Ha R S, Chang P, Gan Y, Yao X, Angelini B, Hibshoosh H, Taback B, Hendon C P. Fully Automated Postlumpectomy Breast Margin Assessment Utilizing Convolutional Neural Network Based Optical Coherence Tomography Image Classification Method. Acad Radiol. 2020 May; 27(5):e81-e86. PMCID: PMC7456393
24. Yao X, Gan Y, Chang E, Hibshoosh H, Feldman S, Hendon C. Visualization and tissue classification of human breast cancer images using ultrahigh-resolution OCT. Lasers Surg Med. 2017 March; 49(3):258-269. PMCID: PMC5368015
25. Bareja R, Mojahed D, Hibshoosh H, Hendon C. Classifying breast cancer in ultrahigh-resolution optical coherence tomography images using convolutional neural networks. Appl Opt. 2022 May 20; 61(15):4458-4462. PMID: 36256284
26. Singh-Moon R P, Marboe C C, Hendon C P. Near-infrared spectroscopy integrated catheter for characterization of myocardial tissues: preliminary demonstrations to radiofrequency ablation therapy for atrial fibrillation. Biomed Opt Express. 2015 Jul. 1; 6(7):2494-2511. PMCID: PMC4505704
27. Singh-Moon R P, Yao X, Iyer V, Marboe C, Whang W, Hendon C P. Real-time optical spectroscopic monitoring of nonirrigated lesion progression within atrial and ventricular
tissues. J Biophotonics. 2019 April; 12(4):e201800144. PMCID: PMC6353711 28. Park S Y, Yang H, Marboe C, Ziv O, Laurita K, Rollins A, Saluja D, Hendon C P. Cardiac endocardial left atrial substrate and lesion depth mapping using near-infrared spectroscopy. Biomed Opt Express. 2022 Apr. 1; 13(4):1801-1819. PMCID: PMC9045901
29. Park S Y, Singh-Moon R, Yang H, Saluja D, Hendon C. Quantification of irrigated lesion morphology using near-infrared spectroscopy. Sci Rep. 2021 Oct. 11; 11(1):20160. PMCID: PMC8505541
30. Park S Y, Singh-Moon R P, Yang H, Hendon C P. Monitoring of irrigated lesion formation with single fiber based multispectral system using machine learning. J Biophotonics. 2022 September; 15(9):e202100374. PMCID: PMC9452461
31. Park S Y, Singh-Moon R P, Wan E Y, Hendon C P. Towards real-time multispectral endoscopic imaging for cardiac lesion quality assessment. Biomed Opt Express. 2019 Jun. 1; 10(6):2829-2846. PMCID: PMC6583339
32. Jogin M, Mohana, Madhulika M S, Divya G D, Meghana R K, Apoorva S. Feature Extraction using Convolution Neural Networks (CNN) and Deep Learning. 2018 3rd IEEE International Conference on Recent Trends in Electronics, Information & Communication Technology (RTEICT). 2018. p. 2319-2323.
33. Suzuki K. Overview of deep learning in medical imaging. Radiol Phys Technol. 2017 September; 10(3):257-273. PMID: 28689314

What is claimed is:

1. A probe for determining at least one structure or at least one characteristic of a uterus, comprising:
a fiber bundle which includes a group of fibers which transmit one or more first electro-magnetic (EM) radiation to at least one portion of a uterus, and another group of fibers which receive one or more second EM radiations exiting the at least one portion which are based on the first EM radiations; and
a hyperspectral imaging camera obtaining at least one image data associated with the second EM radiations to determine the at least one of the structure or the at least one of the characteristic.

2. The probe of claim 1, wherein the probe is an endoscope.

3. The probe of claim 1, wherein the probe is configured to not contact a surface of the uterus to be analyzed while being provided within the uterus.

4. The probe of claim 1, wherein the probe relies on spectral contrast to identify structures and characteristics of a uterus.

5. The probe of claim 1, wherein the fiber bundle emits near infrared wavelengths.

6. The probe of claim 1, wherein the fiber bundle emits near infrared and visible wavelengths.

7. The probe of claim 1, wherein the fiber bundle comprises a minimum of 1000 fibers.

8. The probe of claim 1, wherein the hyperspectral imaging camera is a spectroscopic hyperspectral imaging camera.

9. A system for determining at least one structure or at least one characteristic of a uterus, comprising:
a probe comprising:
a fiber bundle which includes a group of fibers which transmit one or more first electro-magnetic (EM) radiation to at least one portion of a uterus, and another group of fibers which receive one or more second EM radiations exiting the at least one portion which are based on the first EM radiations; and
a hyperspectral imaging camera obtaining at least one image data associated with the second EM radiations to determine the at least one of the structure or the at least one of the characteristic.

10. The system of claim 9, wherein the probe is an endoscope.

11. The system of claim 9, wherein the probe is configured to not contact a surface of the uterus to be analyzed while being provided within the uterus.

12. The system of claim 9, wherein the probe relies on spectral contrast to identify structures and characteristics of a uterus.

13. The system of claim 9, wherein the fiber bundle emits near infrared wavelengths.

14. The system of claim 9, wherein the fiber bundle emits near infrared and visible wavelengths.

15. The system of claim 9, wherein the fiber bundle comprises a minimum of 1000 fibers.

16. The system of claim 9, wherein the hyperspectral imaging camera is a spectroscopic hyperspectral imaging camera.

17. A method for determining structures and characteristics in a uterus, comprising:
illuminating a uterine surface with a fiber bundle in a probe; and
capturing, by a hyperspectral imaging camera in the probe, image data of the illuminated uterine surface.

18. The method of claim 17, wherein the probe is an endoscope.

19. The method of claim 17, wherein the probe is configured to not contact a surface of the uterus to be analyzed while being provided within the uterus.

20. The method of claim 17, wherein the structures and characteristics in the uterus are identified through spectral contrast.

21. The method of claim 17, wherein the illumination by the fiber bundle comprises near infrared wavelengths.

22. The method of claim 17, wherein the illumination by the fiber bundle comprises near infrared and visible wavelengths.

23. The method of claim 17, wherein the probe is configured to contact the uterine surface.

24. The method of claim 17, wherein the hyperspectral imaging camera is a spectroscopic hyperspectral imaging camera.

25. A method for determining structures and characteristics in a uterus, comprising:
imaging a portion of a uterus with a spectroscopic non-contact probe.

26. The method of claim 25, wherein the probe includes a hyperspectral imaging camera.

27. The method of claim 26, wherein the hyperspectral imaging camera is a spectroscopic hyperspectral imaging camera.
